(19) United States
(12) Patent Application Publication
(10) Pub. No.: US 2026/0253700 A1
(43) Pub. Date: Aug. 27, 2026

(54) System and Method for Conversational Personalization of Therapeutic Outputs Using Biomarker-Driven Retrieval-Augmented Generation

(71) Applicant: Médéric Degoy, Perpignan (FR)

(72) Inventor: Médéric Degoy, Perpignan (FR)

(21) Appl. No.: 19/548,019

(22) Filed: Feb. 24, 2026

Related U.S. Application Data
(60) Provisional application No. 63/762,661, filed on Feb. 25, 2025.

(51) Int. Cl. G16H 20/00; G16H 40/63

(52) U.S. Cl. CPC G16H 20/00; G16H 40/63

(57) ABSTRACT

A computer-implemented method enables conversational personalization of therapeutic outputs using biomarker-specific retrieval-augmented generation. Physiological biomarkers and their relational mapping, integral coherence scores, and causal chains are provided as context to a large language model or specialized AI assistants. The system generates tailored explanations, asks clarifying questions, and iteratively refines suggestions based on user responses and updated biomarker data. Specialized assistants, each focused on a subset of biomarkers or categories, interact with the user to modify therapeutic protocols in real time. Live biofeedback can be triggered during dialogue to re-compute features and adapt outputs, achieving highly individualized coherence restoration and health and wellness guidance.

### CROSS-REFERENCE TO RELATED APPLICATIONS

This application claims the benefit of U.S. Provisional Patent Application No. 63/762,661, filed Feb. 25, 2025, the entire contents of which are incorporated herein by reference.

### BACKGROUND OF THE INVENTION

### Field of the Invention

The present invention relates generally to conversational artificial intelligence systems and personalized therapeutic guidance. More particularly, it pertains to methods and systems that employ biomarker-specific retrieval-augmented generation and specialized AI assistants to deliver adaptive, interactive wellness or therapeutic recommendations through multi-turn dialogue.

### Description of Related Art

Conventional conversational AI systems and large language models provide general-purpose responses or retrieve information from static knowledge bases via retrieval-augmented generation (RAG). In health and wellness domains, such systems may access electronic health records or generic medical knowledge but rarely integrate deeply personalized, proprietary physiological biomarkers and their relational structures in real time. Existing approaches lack mechanisms for specialized assistants focused on distinct biomarker categories, live biofeedback triggering during conversation, or direct modification of therapeutic outputs (e.g., protocol modification) based on iterative user dialogue. There remains a need for conversational systems that leverage comprehensive biomarker analysis to provide highly individualized, adaptive therapeutic guidance through dynamic, multi-turn interaction.

### BRIEF SUMMARY OF THE INVENTION

The present invention provides a computer-implemented method and system for conversational personalization of therapeutic outputs using biomarker-specific retrieval-augmented generation. Physiological biomarkers, their relational mapping, their computed data such as energy, entropy or integral coherence scores, and causal chains are provided as context to a large language model or specialized AI assistants. The system generates tailored explanations, asks clarifying questions, and iteratively refines suggestions based on user responses and updated biomarker data. Multiple specialized assistants, each focused on a subset of biomarkers or physiological/psychological categories, interact with the user to refine understanding and propose targeted modifications to therapeutic protocols in real time. Live biofeedback can be triggered during dialogue to re-compute features and adapt outputs, achieving highly individualized coherence restoration and wellness guidance.

### DETAILED DESCRIPTION

The invention is illustrated by way of example and not limitation in the figures of the accompanying drawings, in which like reference numerals indicate similar elements. In particular:
100 designates the Biomarkers and Relational Data Input
110 designates the Context Preparation Module
120 designates the Biomarkers selection
125 designates the automatic selection from the AI assistant specialty
126 designates the manual selection from the AI assistant request
127 designates the manual selection from the interface context or the user request
130 designates the Specialized AI Assistants (plurality)
135 designates the AI assistants output
140 designates the Conversational Loop/Multi-Turn Dialogue
150 designates the User Response Input
160 designates the Therapeutic Output Modification Module
170 designates the Biofeedback Trigger/Live Measurement Interface
180 designates the Updated Biomarker/Feature Re-computation
190 designates the Personalized Therapeutic Output Delivery

The system receives a current set of physiological biomarkers associated with a user (100), together with their relational mapping, integral coherence scores, causal chains, and harmonic qualities derived from prior signal processing.

These data are prepared and formatted as structured context (110) for input to a retrieval-augmented generation system or large language model, with a custom selection of biomarkers (120) repaired primarily according to the AI assistant's specialty, with optional additions of further biomarkers at the request of the AI assistant (125), based on the current interface context (126) or at the user's explicit request (manual selection) (127).

One or more specialized AI assistants (130), each configured to focus on a specific subset of biomarkers or physiological/psychological categories (e.g., energy-related, mind-related, coherence-related), receive the context and generate personalized therapeutic suggestions, explanations, or clarifying questions tailored to their assigned domain.

The assistants interact with the user in a multi-turn conversational loop (140), presenting outputs in natural language and receiving user responses (150).

Based on the conversation, the system modifies therapeutic output parameters in real time (160), including but not limited to: selection or weighting of audio frequencies in harmonic boost mode, re-prioritization of resources and priorities in a protocol, adjustment of guided session content, or alteration of micro-current delivery parameters in a Harmonizer mode.

During the dialogue, the system may trigger live biofeedback measurement via an interface (170) to acquire new physiological data, triggering re-computation of biomarkers, relational features, or coherence scores (180) to update the context and refine subsequent outputs.

The iterative loop continues until the therapeutic protocol achieves a target coherence level, user-defined goal, or sufficient personalization, as determined by updated integral coherence scores or explicit user feedback.

At all turns and at the end, the user is presented with a personalized therapeutic output delivery (audio, visual or interactive) that can be further modified during the conversation (190).

The invention may be embodied in software applications, wearable devices, cloud systems, or hybrid platforms, delivering personalized therapeutic guidance through audio, visual, interactive, and optional micro-current outputs optimized for health issues, wellness, stress management, and coherence restoration.

### BRIEF DESCRIPTION OF THE DRAWINGS

FIG. 1 is a block diagram illustrating an exemplary system for conversational personalization of therapeutic outputs, showing the flow from biomarker and relational data input through biomarker selection logic, context preparation, specialized AI assistants, multi-turn dialogue loop, user response processing, real-time therapeutic output modification, optional live biofeedback triggering, and updated feature re-computation.

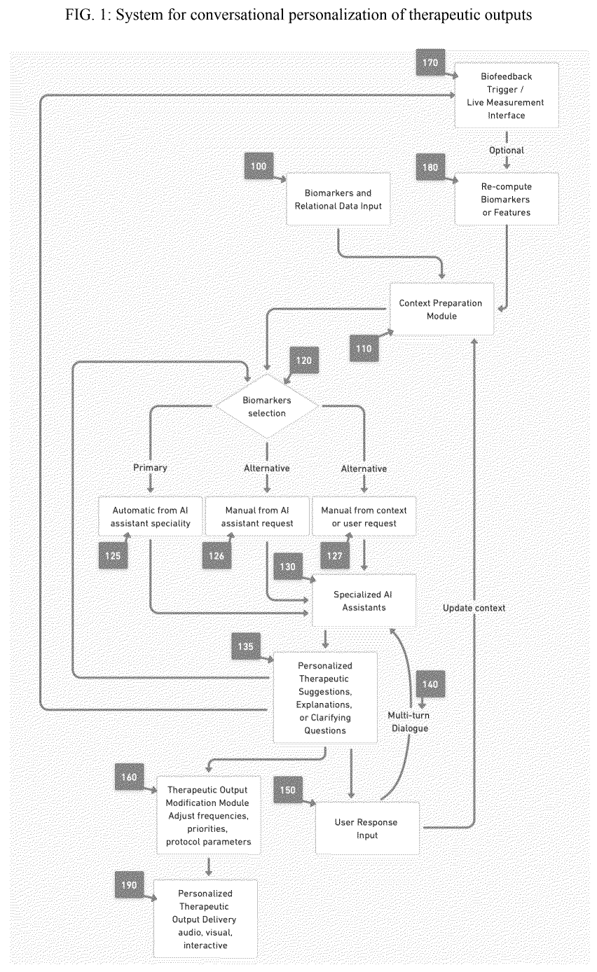

1. A computer-implemented method for conversational personalization of therapeutic outputs, comprising:
obtaining a current set of physiological biomarkers associated with a user, together with their relational mapping, integral coherence scores, and causal chains;
providing said biomarkers, scores, and chains as context to a retrieval-augmented generation system or large language model;
generating, via the model, personalized therapeutic suggestions, explanations, or clarifying questions;
receiving one or more user responses to the questions;
updating the context with the user responses and, if necessary, re-computing or retrieving updated biomarker data;
iteratively refining the model's output in a multi-turn conversational loop; and
modifying at least one therapeutic output parameter in real time based on the refined output.

2. The method of claim 1, wherein multiple specialized AI assistants are employed, each assistant configured to receive and process a subset of biomarkers, relational mapping, or coherence scores corresponding to a specific physiological, energetic, or psychological category.

3. The method of claim 2, wherein the specialized assistants interact with the user in parallel or sequence to refine understanding, ask category-specific clarifying questions, or propose targeted modifications to the therapeutic protocol.

4. The method of claim 1, further comprising triggering real-time biofeedback measurement during the conversation, re-computing one or more biomarkers or relational features based on the measurement, and updating the model context accordingly.

5. The method of claim 1, wherein the therapeutic output modification includes at least one of: adjusting the selection or weighting of audio frequencies in a harmonic boost mode, re-prioritizing resources and priorities in a protocol, modifying guided session content, or altering micro-current delivery parameters in a connected device.

6. A system for conversational personalization of therapeutic outputs, comprising:
a processor;
memory storing instructions that, when executed, cause the processor to obtain physiological biomarkers, relational mapping, coherence scores, and causal chains;
provide said data as context to a retrieval-augmented generation system or large language model;
generate personalized suggestions or questions;
receive user responses;
update context iteratively; and
modify therapeutic output parameters in real time based on the conversational loop.

7. The system of claim 6, further comprising a plurality of specialized AI assistants, each configured to handle a subset of biomarker categories or relational aspects.

8. The system of claim 6, further comprising a biofeedback interface configured to trigger live physiological measurements during the conversation and update biomarker data in the model context.

9. The method of claim 1, wherein the model generates explanations of biomarker states, coherence scores, relational influences, or therapeutic implications in natural language tailored to the user's current context and knowledge level.

10. The method of claim 1, wherein the conversational loop continues until the therapeutic protocol achieves a target coherence level or user-defined goal, as determined by updated integral coherence scores or user feedback.
